(19) United States
(12) Plant Patent Application Publication
(10) Pub. No.: US 2015/0195968 P1
(43) Pub. Date: Jul. 9, 2015

(54) Apple Tree Named Howell TC3

(50) ;

(71) Applicant: William Edward Howell, Prosser, WA (US)

(72) Inventor: William Edward Howell, Prosser, WA (US)

(21) Appl. No.: 14/544,402

(22) Filed: Dec. 31, 2014

Related U.S. Application Data
(60) Provisional application No. 61/924,174, filed on Jan. 6, 2014.

(51) Int. Cl. A01H 5/00

(52) U.S. Cl. PLT/161

(57) ABSTRACT

A new and distinct apple tree is disclosed. ‘Howell TC3’ is notable for its distinctive fruit, which has yellow skin and red flesh with a sweet/tart flavor.

### CROSS-REFERENCE TO RELATED APPLICATIONS

None.

### STATEMENT REGARDING FEDERALLY SPONSORED RESEARCH OR DEVELOPMENT

None.

### LATIN NAME OF THE GENUS AND SPECIES OF THE PLANT CLAIMED

Malus Domestica.

### VARIETY DENOMINATION

‘Howell TC3’

### BACKGROUND OF THE INVENTION

The new variety of apple tree ‘Howell TC3’ was created in the course of a planned breeding program carried out in Prosser, Wash. ‘Howell TC3’ originated as a result of an open pollination cross in 2001 of ‘Airlie Red Flesh’ (seed parent) with the likely pollen parent being ‘Honeycrisp’ (U.S. Plant Pat. No. 7,197, expired). ‘Howell TC3’ was selected in 2010 as a single plant from among a population of seedlings derived from the parents, and was selected for its quality of fruit and the red color of the fruit flesh. It is distinguished from its parents by its yellow skin and red flesh color and by its flavor. Asexual propagation by budding was first carried out in Prosser, Wash. The variety has since been observed over a number of asexually propagated generations, and has been found to remain true to type.

### BRIEF SUMMARY OF THE INVENTION ‘Howell TC3’ is a new and distinct apple tree notable for its distinctive fruit, which has a red flesh and a sweet/tart flavor.

### BRIEF DESCRIPTION OF THE PHOTOGRAPH

FIG. 1 shows the blossom from the tree of the new cultivar;

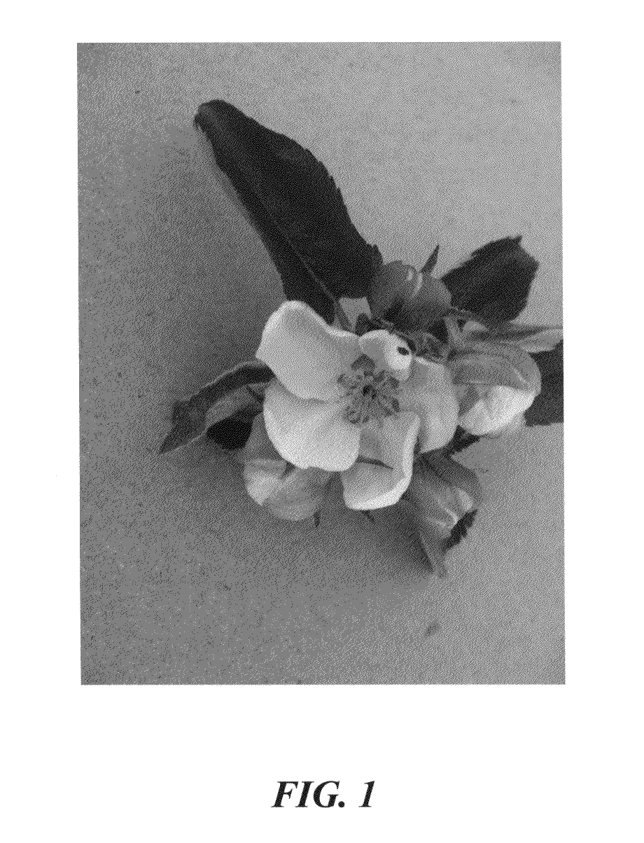

FIG. 2 the fruit of the new cultivar on the tree; and

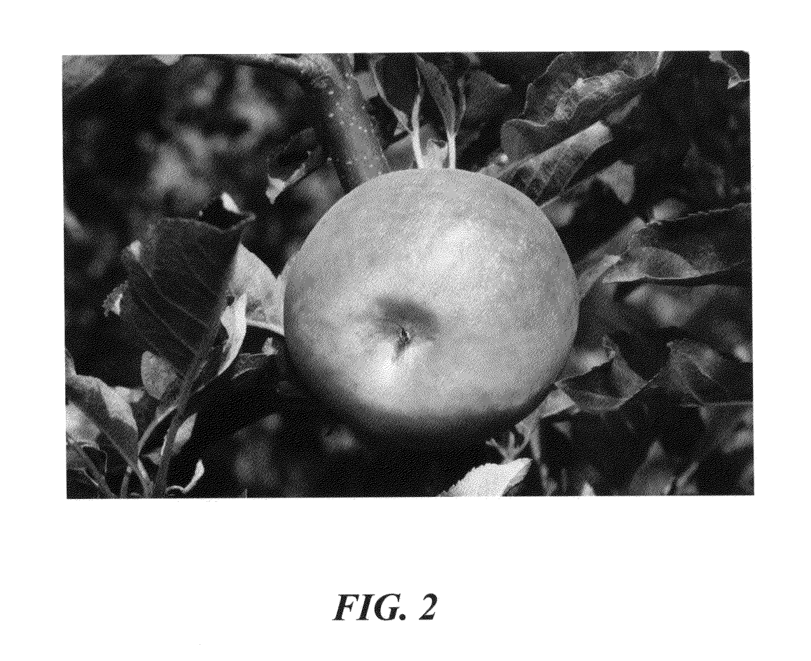

FIG. 3 shows the flesh of the fruit of the new cultivar.

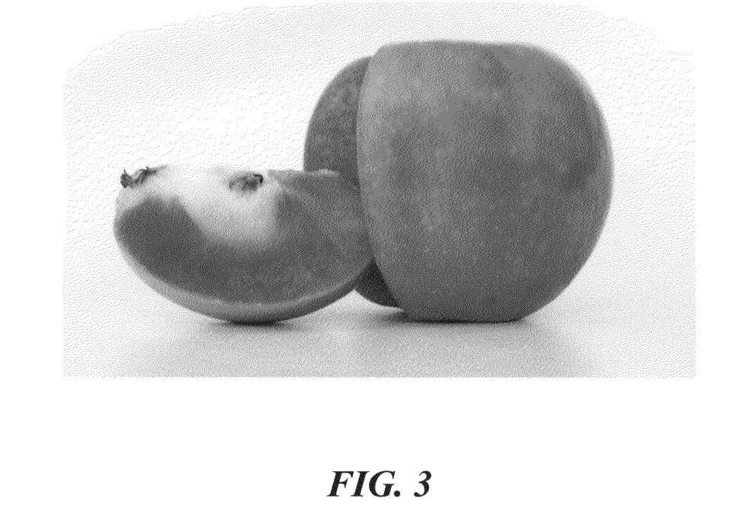

### DETAILED BOTANICAL DESCRIPTION

The following detailed botanical description is based on observations made during the 2013 growing season at Prosser, Wash. All colors are described according to The Royal Horticultural Society Colour Chart as published in 2001. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average.
Tree: Vigor — semi-vigorous; Size — Height 9 feet; Spread 4 feet; Trunk — Diameter at 30 cm above graft union 2.54 cm; Bark texture — smooth; Bark coloration — Gray to reddish-brown.
Branch: Size — Length 81.3 cm; Diameter 1.27 cm at 1 m above ground; Crotch angle relative to trunk — 60°; Color — Gray to reddish-brown.
Dormant one-year-old shoot: Size — diameter 0.95 cm, length 71.1 cm; Color — Gray to reddish-brown; Internode length — 3.8 cm.
Flowers: Bud shape — Cone; Bud length — 0.95 cm; Bud Diameter — 0.48 cm; Color at balloon stage — Dark pinkish red with white areas; Diameter of open flower — 3.81 cm; Petal — Quantity per flower 5; Margin — smooth; Relative position of petal margin — overlapping; Petal length — 2.54 cm; Petal width — 2.54 cm; Color of upper surface — White; Color of lower surface — Whitish Pink; Pistil size — 1.91 cm; Anther quantity — 21 to 25; Size — 0.32 cm; Color of pollen — Yellow; Stigma size — 0.16 cm; Stigma color — Whitish-Yellow; Style size — 1.9 cm; Style color — Greenish Brown; Ovary color — Green; Bloom period — First bloom April 22, full bloom April 30 in Prosser, Wash.
Pedicel: Length — 1.9 cm; Diameter — 0.16 cm; Color — Green.
Sepals: Quantity — 5; Color — Green; Size — 1.9 cm; Shape — Lance.
Leaf: Shape — Ovate; Attitude in relation to shoot — nearly vertical; Blade margin — serrated; Apex — Acuminate; Base shape — Rounded; Size — length 10.2 cm, width 5.7 cm; Color of upper surface — dark green; Color of lower surface — green.
Petiole: Length — 2.86 cm; Diameter — 0.32 cm
Fruit: Size — diameter 7.62 cm; Weight — 0.45 lbs; General shape in profile — Rounded; Ribbing absent; Crowning at calyx end — Moderate; Stalk — length 2.54 cm, width 0.32 cm; Depth of stalk cavity 1.91 cm; Width of stalk cavity 0.64 cm; Size of lenticels 0.08 cm; Ground color of skin — Yellow; Over color of skin — Yellow; Pattern of over color — Mottled from red flesh showing through skin; Flesh is crisp and very juicy; Flesh color — Red; Brix — 15° ; Quantity of seeds 8-10; Shape of seed — Acuminate; Seed color Dark Brown.
Disease Resistance/Susceptibility: None noted.
Use: Fresh Market and processing.

1. We claim a new and distinct apple tree substantially as shown and described herein.
